(19) United States
(12) Patent Application Publication
(10) Pub. No.: US 2010/0184107 A1
(43) Pub. Date: Jul. 22, 2010

(54) PROTEIN TRAFFICKING

(76) Inventors: Robert J. Lefkowitz, Durham, NC (US); Jonathan D. Violin, Durham, NC (US)

Correspondence Address: NIXON & VANDERHYE, PC, 901 NORTH GLEBE ROAD, 11TH FLOOR, ARLINGTON, VA, 22203, US

(21) Appl. No.: 12/449,650

(22) PCT Filed: Feb. 21, 2008
(86) PCT No.: PCT/US08/02257

§ 371 Date: Mar. 12, 2010

Related U.S. Application Data
(60) Provisional application No. 60/902,353, filed on Feb. 21, 2007.

(51) Int. Cl. C12Q 1/48; C12Q 1/02; C12N 5/00

(52) U.S. Cl. 435/15; 435/29; 435/325

(57) ABSTRACT

The present invention relates, in general, to protein trafficking, and, in particular, to a method of measuring protein trafficking to and from a plasma membrane.

This application claims priority from Provisional Application No. 60/902,353, filed Feb. 21, 2007, the entire content of which is incorporated by reference.

This invention was made with government support under Grant Nos. 2RO1HL016037-33 and 5RO1HL070631-04 awarded by the National Institutes of Health. The government has certain rights in the invention.

### TECHNICAL FIELD

The present invention relates, in general, to protein trafficking, and, in particular, to a method of measuring protein trafficking to and/or from a plasma membrane.

### BACKGROUND OF THE INVENTION

Protein trafficking is an important regulatory mechanism for the function of many drug targets. The translocation to and from the plasma membrane serves to regulate access of a protein to the extracellular environment. For many drug targets, such as G protein coupled receptors (GPCRs), receptor tyrosine kinases (RTKs), and ion channels, cell surface expression correlates with physiological and pathophysiological functions.

Various methods have been developed for measuring cell surface expression of such proteins, including radioligand binding, flow cytometry using fluorescent antibodies, and imaging of target protein fusion to fluorescent proteins. However, these methods are expensive and difficult and require both technical expertise and highly specialized equipment.

Previous work has shown that fluorescent proteins targeted to the plasma membrane can undergo fluorescent resonance energy transfer (FRET) with each other (Zacharias et al, Science 296(5569):913-16 (2002)). It has also been found that a plasma membrane-targeted fluorescent protein can undergo FRET when a second fluorescent fusion protein translocated from cytosol to plasma membrane (Violin et al, J. of Cell Biol. (2003); U.S. Patent Application 20050026234).

The present invention provides a simple method for measuring cell surface expression of a protein using, for example, FRET or bioluminescent resonance energy transfer (BRET). This method is applicable to any protein that translocates to and/or from the plasma membrane, whether or not well characterized.

### SUMMARY OF THE INVENTION

The present invention relates generally to protein trafficking. More specifically, the invention relates to a method of measuring protein trafficking to and/or from a plasma membrane.

Objects and advantages of the present invention will be clear from the description that follows.

### BRIEF DESCRIPTION OF THE DRAWINGS

FIGS. 1A-1D. The method of the invention relies on a high effective concentration that results from molecules confined to two dimensions. Donor molecule, cyan fluorescent protein mCFP, is fused to GPCR, which is bound to or embedded in the plasma membrane. Acceptor molecule, yellow fluorescent protein mYFP, fused to a sequence encoding lipid modification, myristoyl and palmitoyl, targets to the plasma membrane (FIG. 1A). When MyrPalm-mYFP is expressed to sufficiently high levels, the probability of the GPCR-mCFP being sufficiently close to a MyrPalm molecule to permit FRET increases (FIG. 1B). FRET is measured in single cells transiently transfected with MyrPalm-mYFP; FRET increases as the concentration (measured by fluorescence intensity) of YFP increases (FIGS. 1C, 1D).

FIGS. 2A-2D. When GPCRs are stimulated, receptors internalize by trafficking from plasma membrane to intracellular vesicles thereby physically separating GPCR-mCFP from MyrPalm-mYFP (FIG. 2A, before internalization; FIG. 2B, after internalization). This is demonstrated microscopically. FIG. 2C shows colocalization of β₂AR-mCFP and MyrPalm-mYFP before stimulation. FIG. 2D shows separation of β₂AR-mCFP and MyrPalm-mYFP after stimulation.

FIG. 3. The physical separation of β₂AR-mCFP and MyrPalm-mYFP results in loss of FRET. Shown is one edge of two abutting cells.

FIGS. 4A-4D. FRET can be quantified over time, permitting quantitation of internalization kinetics 1 μM isoproterenol results in loss of FRET with half time of approximately 5 minutes (FIG. 4A). A panel of β₂AR ligands at receptor-saturating doses results in internalization with variable kinetics (FIG. 4B). Image of cell line after isoproterenol treatment, showing cell surface and internalized β₂AR-mCFP and plasma membrane-limited mYFP and FRETc (FIG. 4C). Rate of FRET loss after stimulation, as measured by a monoexponential model with rate k(obs) and maximum loss E(min); k(obs) used to compare ligand efficiency for internalization (FIG. 4D).

FIGS. 5A and 5B. The assay method of the invention used with SDF-mediated internalization of CXCR4 (FIG. 5A) and Angiotensin II type 1 receptor (FIG. 5B).

### DETAILED DESCRIPTION OF THE INVENTION

The present invention relates to a simple and rapid method for measuring protein trafficking to and/or from a plasma membrane. This invention provides a fluorescence-based or bioluminescence-based approach to measuring protein movement without the use of radioactivity or antibodies. This method can be applied to any protein moving to and/or from the plasma membrane, including, but not limited to, receptor tyrosine kinases, and it can be used to examine the function of regulators of membrane trafficking.

The present method relies on the very high effective concentration that results from molecules (paired combinations of fluorescent proteins, bioluminescent proteins or small molecules that undergo resonance energy transfer) confined to two dimensions (Kenworthy & Edidin, J. Cell Biol. 142(1): 69-84, 1998). In accordance with the method, a fluorescent, luminescent, or bioluminescent donor molecule (e.g., the cyan fluorescent protein mCFP, the bioluminescent renilla luciferase, or lanthanide chelates) is fused to a target protein that, at any point, is bound to or embedded in the plasma membrane. Examples of target proteins include, but are not limited to, GPCRs (Jacoby et al, Chem Med Chem 1(8):761-82, 2006), including the β₂-adrenergic receptor (β₂AR), Angiotensin II type 1 receptor, CXCR4, CCR7, RTKs, including epidermal growth factor receptor (EGFR), platelet-derived growth factor receptor (PDGFR), and insulin receptor (IR), as well as ion channels, including the cystic fibrosis transmembrane conductance regulator (CFTR) and voltage-dependent calcium channels (VDCC). In the same cells, a fluorescent or fluorescent quenching acceptor molecule (e.g., the yellow fluorescent protein mYFP, the red fluorescent protein tdTomato), is constitutively targeted to plasma membrane. For example, the acceptor molecule can be fused to a short sequence encoding lipid modification, such as myristoyl and palmitoyl (MyrPalm-mYFP, Zacharias et al, Science 296(5569):913-16 (2002)), that automatically targets to plasma membrane. Alternative methods for membrane-targeting of the acceptor fluorophore include other protein modifications (e.g. polybasic domains or isoprenylation), attaching fluorescent proteins to transmembrane proteins, or use of hydrophobic fluorescent dyes such as BODIPY FL. When the membrane-targeted acceptor molecule is introduced, the probability of the donor/target fusion being sufficiently close to an acceptor molecule to permit resonance energy transfer increases dramatically. Resonance energy transfer will occur in direct proportion to the concentration of acceptor fluorophore.

The invention is exemplified below with reference to receptors which, when stimulated appropriately, as by some ligands, internalize by trafficking from plasma membrane to intracellular vesicles. When this occurs, the donor/receptor fusion is physically separated from the acceptor molecule, which remains limited to plasma membrane. This physical separation results in loss of resonance energy transfer (which can be corrected for spectral bleedthrough (Gordon et al, Biophysical Journal 74(5):2702-13 (1998)). Since no resonance energy transfer is detected on internalized vesicles, the resonance energy transfer signal for a given expression level of donor/receptor fusion and acceptor molecule corresponds to the amount of receptor in the plasma membrane. To keep these expression levels constant over time and between experimental conditions, the use of clonal cells stably transfected with both membrane-targeted acceptor and donor/receptor fusion is preferred. The method is not limited to β-arrestin-dependent internalization but can be used to measure all mechanisms of internalization. Resonance energy transfer can be quantified over time, permitting quantitation of internalization kinetics.

It has been observed that receptor internalization can be increased by overexpression of 3-arrestin (Zhang et al, J. Biol. Chem. 271(31):18302-5 (1996)). Thus, β-arrestin overexpression can be used to increase the response of the method described herein. For example, to amplify the internalization of the β₂AR detected by this assay, β-arrestin can be overexpressed by transfection, resulting in a larger percentage of β₂AR internalization from the cell surface.

As indicated above, the response of the present method depends on the concentrations and stoichiometric ratios of the donor and acceptor molecules. Thus, the present method is the most robust when both the membrane-bound acceptor and the trafficking donor fusion are present at constant levels, either through stable transfection in the case of genetically encoded reporters or careful titration for fluorescent dyes. Genetically encoded reporters are preferred because of low cost and reliable signal. While the present method can be carried out with any equipment capable of detecting resonance energy transfer, including fluorimeters, microscopes, high-content imaging systems, and plate readers, use of a high-throughput plate reader is preferred.

The present method can be used, for example, to measure the rate and amount of internalization of, for example, GPCRs, including the β₂-adrenergic receptor (β₂AR), Angiotensin II type 1 receptor, CXCR4, and CCR7, RTKs, including epidermal growth factor receptor (EGFR), platelet-derived growth factor receptor (PDGFR), and insulin receptor (IR), as well as ion channels, including the cystic fibrosis transmembrane conductance regulator (CFTR) and voltage-dependent calcium channels (VDCC). It will be appreciated from a reading of this disclosure that the method described can be used to screen for and characterize GPCR ligands, including full agonists, antagonists, partial agonists, inverse agonists. When paired with a second assay of GPCR function such as second messenger, signal transduction, or cellular changes, this assay can be used to screen for and characterize biased GPCR ligands (see U.S. Provisional Application No. 60/838,474). In addition, this method is applicable to other trafficking proteins, including receptor tyrosine kinases, such as EGFR. Importantly, this method can be used to measure the trafficking of target proteins both to and from the plasma membrane. While the invention is described, at least in part, with reference to GPCRs internalizing from plasma membrane, application of the method to trafficking to the membrane is extremely useful, for example, in screening for and characterizing compounds that promote proper trafficking of cystic fibrosis CFTR mutants which normally fail to reach the plasma membrane. This method can also be used to evaluate components of trafficking machinery, such as proteins involved in vesicle budding or fusion (Drake et al, Circ. Res. 99(6): 570-82, 2006). Furthermore, this approach can be used to assess G protein-coupled receptor kinase (GRK) and beta-arrestin functions.

Certain aspects of the invention are described in greater detail in the non-limiting Examples that follow. (See also US Application Numbers 20050026234 and 20060199226.).

### Example 1

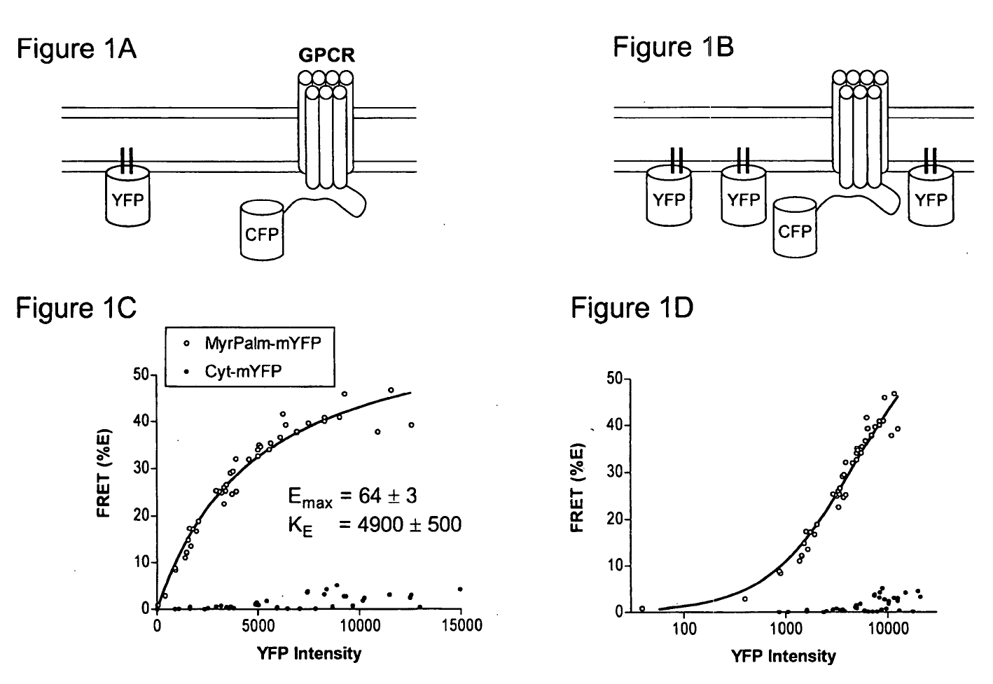

A donor molecule, the cyan fluorescent protein mCFP, is fused to a target protein, the GPCR β₂AR. Separately, an acceptor molecule, in this case the yellow fluorescent protein mYFP, is constitutively targeted to plasma membrane by fusion to a short sequence encoding lipid modification (MyrPalm-mYFP, Zacharias et al, Science 296(5569):913-16 (2002)). These two constructs are co-transfected into cells, resulting in colocalization of the MyrPalm-mYFP and β₂AR-mCFP in the plasma membrane, as illustrated in FIG. 1A. As illustrated in FIG. 1B, with increasing expression of MyrPalm-mYFP, the probability of the GPCR-mCFP being sufficiently close to a MyrPalm-mYFP molecule to permit FRET (less than 10 nm) increases dramatically. This is demonstrated by transfecting a GPCR-mCFP (that is, (β₂AR-mCFP), selecting for stable transfectants with appropriate antibiotic (here, G418), and isolating a clonal cell line in which each cell expresses equal density of β₂AR-mCFP, as measured by fluorescence. MyrPalm-mYFP is then transiently transfected into these cells, yielding a population of cells with constant concentration of β₂AR-mCFP but variable concentration of MyrPalm-mYFP. FRET is measured in single cells with a CCD-based, filter equipped epi-fluorescence microscope (Violin et al, J. Biol. Chem 281(29):20577-88 (2006)); FRET increases as the intensity of YFP increases (FIGS. 1C and 1D).

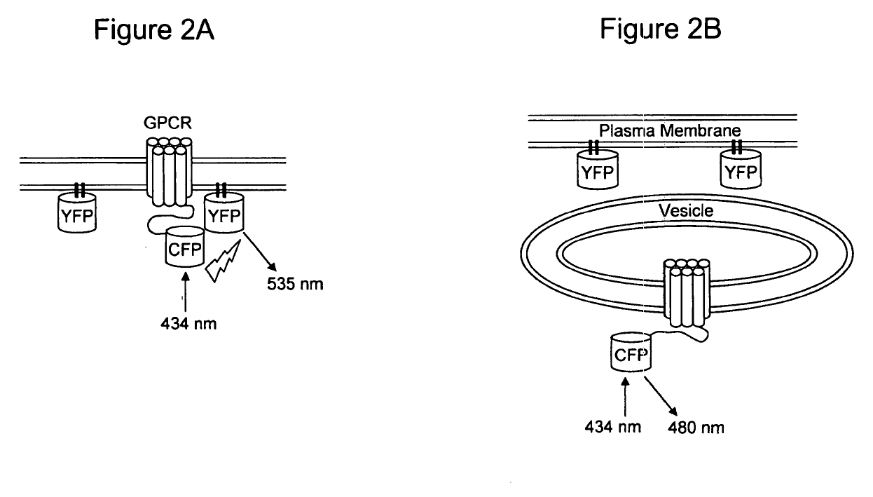

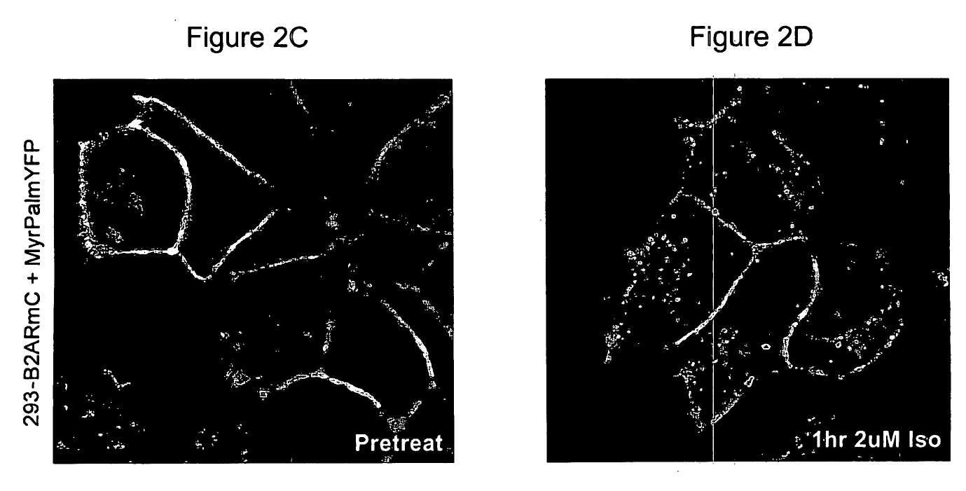

Following ligand stimulation, GPCRs internalize by trafficking from plasma membrane to intracellular vesicles. The GPCR-mCFP is thereby physically separated from the MyrPalm-mYFP, which remains limited to plasma membrane (illustrated in FIG. 2A before internalization, FIG. 2B after internalization). Microscope-based acquisition of filter-selected cyan and yellow images (FIG. 2C) show colocalization of β₂AR-mCFP (green) and MyrPalm-mYFP (red) before stimulation and separation of β₂AR-mCFP and MyrPalm-mYFP to intracellular vesicles and plasma membrane, respectively, after stimulation for 1 hour with 2 μM isoproterenol (FIG. 2D).

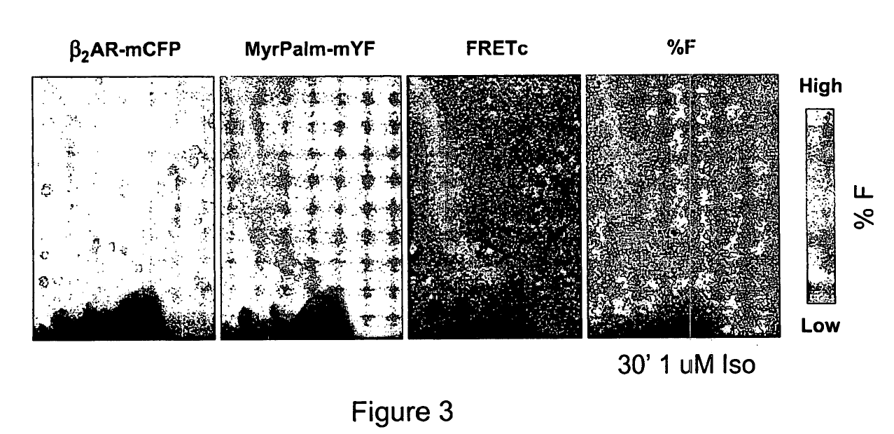

This physical separation also results in loss of FRET, as shown in FIG. 3, which shows one edge of two abutting cells. After 30 minutes of stimulation with 1 μM isoproterenol, β₂AR-mCFP (cyan image) is seen in plasma membrane and vesicles, whereas MyrPalm-mYFP (yellow image) is only seen in plasma membrane. FRETc (red image), the FRET signal corrected for spectral bleedthrough (Violin et al, J. Biol. Chem 281(29): 20577-88, 2006), is found where the two proteins colocalize but not in the vesicles. This is also illustrated by an image of % F, a pseudocolor scale of the ratio FRETc intensity to CFP intensity, ranging from low % F on vesicles (blue) to high % F on plasma membrane (red). Since no FRET is detected on internalized vesicles, the FRET signal for a given expression level of GPCR-mCFP and MyrPalm-mYFP corresponds to the amount of receptor in the plasma membrane.

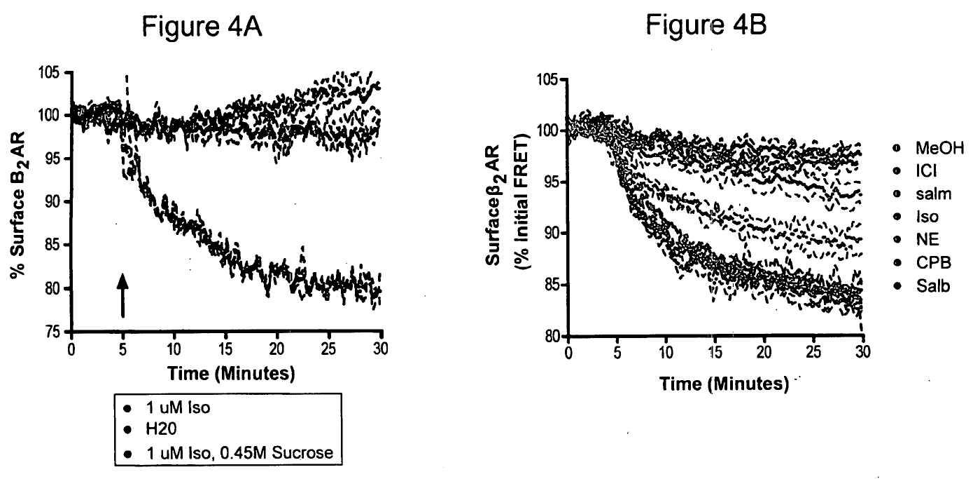

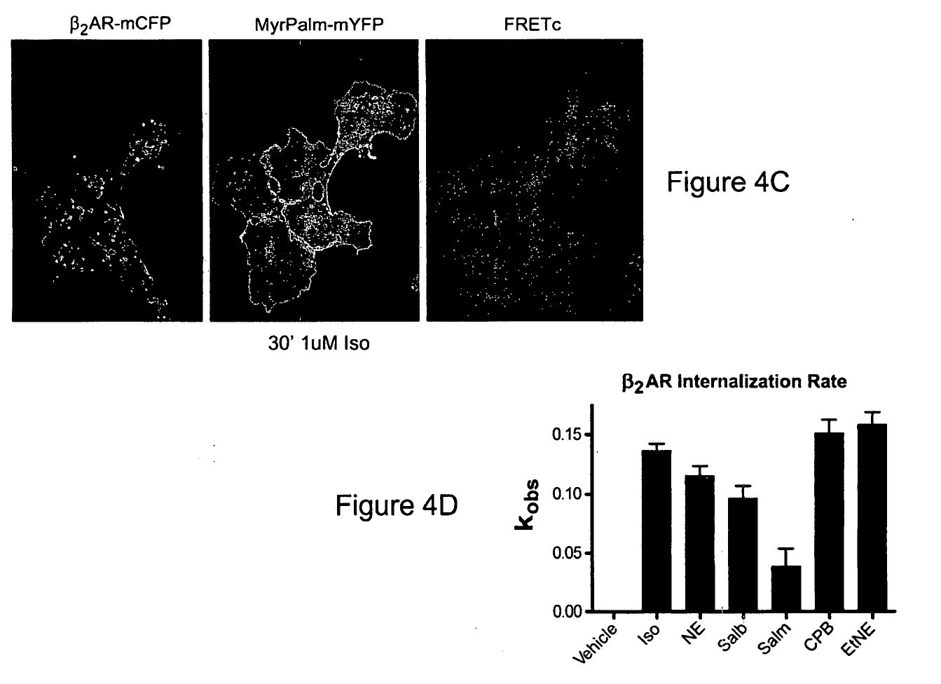

Quantification of FRET over time permits quantitation of internalization kinetics in live cells. 1 μM isoproterenol results in a loss of FRET (% surface β₂AR) with a half-time of approximately 5 minutes (FIG. 4A). This loss of FRET is not seen with addition of buffer alone, or when isoproterenol-stimulated internalization is blocked by 0.45M sucrose. A panel of β₂AR ligands including an antagonist (ICI-118,551), partial agonists (salmeterol, salbutamol, norepinephrine, and cyclopentylbutanephrine), and a full agonist (isoproterenol) at receptor-saturating doses causes internalization with variable kinetics (FIG. 4B), illustrating that this assay can discern variations in ligand efficacy for internalization. A sample image of the cell line used in these experiments is shown after 30 minutes of 1 μM isoproterenol treatment, showing both cell surface and internalized β₂AR-mCFP but plasma membrane-limited mYFP and FRETc (FIG. 4C). One simple measurement of this assay is the rate of FRET loss after stimulation, as measured by a monoexponential model with rate k(obs) and maximum loss E(min). The k(obs) value can be used to compare ligand efficacy for internalization (FIG. 4D).

### Example 2

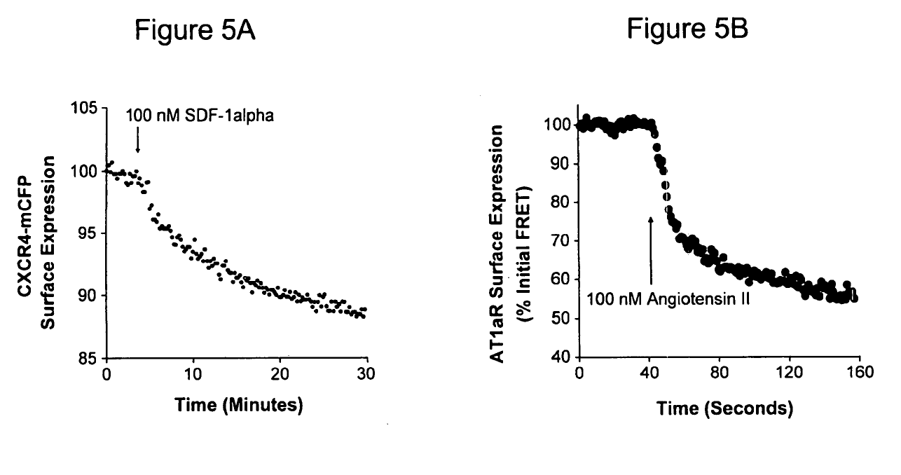

The assay described in Example 1 has also been used with other receptor systems, including SDF-mediated internalization of the chemokine receptor CXCR4 (FIG. 5A) and the Angiotensin II type 1 receptor (AT1_{A}R)(FIG. 5B).

CXCR4-mCFP plasmid was generated and transfected into U2-osteosarcoma cells along with MyrPalm-mYFP. Cells expressing both constructs were located on a FRET-capable microscope (Violin et al, J. Biol. Chem 281(29): 20577-88, 2006), and FRET, normalized to 100%, was measured over time as surface expression of CXCR4-mCFP (FIG. 5A). After 5 minutes, 100 nm SDF-1α was added to the cells, and CXCR4 internalization was reported as the percent of remaining surface expression (% of initial FRET).

Similarly, AT1_{A}R-mCFP plasmids were generated and transfected into HEK-293 cells along with MyrPalm-mYFP. Cells expressing both constructs were located, and FRET, normalized to 100%, was measured over time as surface expression of AT1_{A}R-mCFP (FIG. 5B). after 8 minutes, 100 nM AngII was added to the cells, and AT1_{A}R internalization was reported as the percent of remaining surface expression (% of initial FRET).

All documents and other information sources cited above are hereby incorporated in their entirety by reference.

What is claimed is:

1. A method of monitoring movement of a target protein to or from a plasma membrane comprising contacting a cell comprising:
a) a first member of a pair of molecules that undergo resonance energy transfer, which first member constitutively targets to the plasma membrane of said cell, and
b) a second member of a pair of molecules that undergo resonance energy transfer, which second member is fused to a target protein, wherein said target protein can bind to or embedded within the plasma membrane of said cell,
wherein resonance energy transfer between said first and second members occurs when said first and second members are sufficiently close,
with an agent that stimulates movement of said target protein to or from said plasma membrane,
wherein said movement is monitored by measuring a decrease or increase in resonance energy transfer between said first and second members, an increase in said resonance energy transfer being indicative of movement of said target protein to said plasma membrane and a decrease in said resonance energy transfer being indicative of movement of said target protein away from said plasma membrane.

2. The method according to claim 1 wherein said first and second members are fluorescent or bioluminescent.

3. The method according to claim 1 wherein said first member is an acceptor molecule and said second member is a donor molecule.

4. The method according to claim 3 wherein said acceptor molecule is mYFP and said donor molecule is mCFP.

5. The method according to claim 4 wherein said mYFP is fused to MyrPalm.

6. The method according to claim 1 wherein said target protein is a G-protein coupled receptor (GPCR).

7. The molecule according to claim 6 wherein said GPCR is β₂-adrenergic receptor.

8. The method according to claim 1 wherein said target protein is Angiotensin II type 1 receptor, CxCR4 or CCR7.

9. The method according to claim 1 wherein said target protein is a receptor tyrosine kinase.

10. The population according to claim 1 wherein said target protein is an ion channel.

11. The method according to claim 1 wherein said first member is fused to a polybasic domain or a transmembrane protein.

12. The method according to claim 1 wherein said agent is a ligand that stimulates internalization of said target protein.

13. A population of cloned cells stably transfected with a sequence encoding a first member of a pair of molecules that undergo resonance energy transfer, which first member constitutively targets to the plasma membrane of said cells, and a sequence encoding a second member of said pair of molecules fused to a target protein.

14. The population according to claim 13 wherein said target protein is a receptor.

15. The population according to claim 14 wherein said receptor is a GPCR.

16. The population according to claim 15 wherein said GPCR is β₂-adrenergic receptor.
